(19) United States
(12) Patent Application Publication
(10) Pub. No.: US 2025/0319107 A1
(43) Pub. Date: Oct. 16, 2025

(54) USE OF CANNABIDIOL AND CLOBAZAM IN THE TREATMENT OF CHILDHOOD-ONSET EPILEPSY SYNDROMES

(71) Applicant: GW Research Limited, Cambridgeshire (GB)

(72) Inventors: Volker Armin KNAPPERTZ, Carlsbad, CA (US); Daniel Adam CHECKETTS, Cambridgeshire (GB)

(21) Appl. No.: 18/548,003

(22) PCT Filed: Feb. 22, 2022
(86) PCT No.: PCT/GB2022/050476

§ 371 Date: Aug. 25, 2023

(30) Foreign Application Priority Data
Feb. 25, 2021 (GB) 2102694.3

(51) Int. Cl. A61K 31/00; A61K 31/551; A61K 36/185; A61K 45/06; A61P 25/08

(52) U.S. Cl. CPC A61K 31/658; A61K 31/551; A61K 36/3482; A61K 45/06; A61P 25/08

(57) ABSTRACT

The present invention relates to the use of cannabidiol (CBD) in the treatment of patients with childhood-onset epilepsy syndromes who are concurrently taking the antiepileptic drug clobazam. When CBD is used in combination with clobazam caution should be taken. The patient may need to be cautioned and/or monitored for side effects of a drug-drug interaction 5 between the two medications. In particular the patient should be cautioned and/or monitored for the occurrence of pneumonia. In such a situation the dose of either the CBD and/or the clobazam may be required to be reduced.

### CROSS-REFERENCE TO RELATED APPLICATIONS

This application is a U.S. National Phase application, filed under 35 U.S.C. § 371 (c), of International Application No. PCT/GB2022/050476, filed Feb. 22, 2022, which claims priority to, and the benefit of, United Kingdom Patent Application No. 2102694.3, filed Feb. 25, 2021. Each of these documents is incorporated by reference herein in its entirety for all purposes.

### FIELD OF THE INVENTION

The present invention relates to the use of cannabidiol (CBD) in the treatment of patients with childhood-onset epilepsy syndromes who are concurrently taking the antiepileptic drug clobazam. When CBD is used in combination with clobazam caution should be taken. The patient may need to be cautioned and/or monitored for side effects of a drug-drug interaction between the two medications. In particular the patient should be cautioned and/or monitored for the occurrence of pneumonia. In such a situation the dose of either the CBD and/or the clobazam may be required to be reduced.

In a further embodiment the CBD used is in the form of a highly purified extract of cannabis such that the CBD is present at greater than 95% of the total extract (w/w) and the cannabinoid tetrahydrocannabinol (THC) has been substantially removed, to a level of not more than 0.15% (w/w).

More preferably the CBD used is in the form of a botanically derived purified CBD which comprises greater than or equal to 98% (w/w) CBD and less than or equal to 2% (w/w) of other cannabinoids. More preferably the other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AEDs). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood-onset epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from, an investigation into the type of seizures that the patient is experiencing is undertaken. Clinical observations and electroencephalography (EEG) tests are conducted, and the type(s) of seizures are classified according to the ILAE classification.

Generalised seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a Bilateral convulsive seizure, which is the proposed terminology to replace Secondary Generalised Seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Childhood onset epilepsy syndromes often present with many different types of seizure and identifying the types of seizure that a patient is suffering from is important as many of the standard AEDs are targeted to treat or are only effective against a given seizure type/sub-type.

One such childhood onset epilepsy syndrome is Lennox-Gastaut syndrome (LGS). LGS is a severe form of epilepsy, where seizures usually begin before the age of 4. Seizure types, which vary among patients, include tonic (stiffening of the body, upward deviation of the eyes, dilation of the pupils, and altered respiratory patterns), atonic (brief loss of muscle tone and consciousness, causing abrupt falls), atypical absence (staring spells), and myoclonic (sudden muscle jerks). There may be periods of frequent seizures mixed with brief, relatively seizure-free periods.

Seizures in LGS are often described as “drop seizures”. Such drop seizures are defined as an attack or spell (atonic, tonic or tonic-clonic) involving the entire body, trunk or head that led or could have led to a fall, injury, slumping in a chair or hitting the patient's head on a surface.

Most patients with LGS experience some degree of impaired intellectual functioning or information processing, along with developmental delays, and behavioural disturbances.

LGS can be caused by brain malformations, perinatal asphyxia, severe head injury, central nervous system infection and inherited degenerative or metabolic conditions. In 30-35% of cases, no cause can be found.

Another childhood onset epilepsy syndrome is Dravet syndrome. Onset of Dravet syndrome almost always occurs during the first year of life with clonic and tonic-clonic seizures in previously healthy and developmentally normal infants (Dravet, 2011). Symptoms peak at about five months of age. Other seizures develop between one and four years of age such as prolonged focal dyscognitive seizures and brief absence seizures.

Dravet syndrome patients suffer both focal and generalised seizures and may also experience atypical absence seizures, myoclonic absence seizures, atonic seizures and non-convulsive status epilepticus.

Seizures progress to be frequent and treatment-resistant, meaning that the seizures do not respond well to treatment. They also tend to be prolonged, lasting more than 5 minutes. Prolonged seizures may lead to status epilepticus, which is a seizure that lasts more than 30 minutes, or seizures that occur in clusters, one after another.

Prognosis is poor and approximately 14% of children die during a seizure, because of infection, or suddenly due to uncertain causes, often because of the relentless neurological decline. Patients develop intellectual disability and life-long ongoing seizures. Intellectual impairment varies from severe in 50% patients, to moderate and mild intellectual disability each accounting for 25% of cases.

A further childhood onset epilepsy syndrome is tuberous sclerosis complex (TSC), these patients experience a range of different seizure types. TSC is a genetic disorder that causes mainly benign tumours to develop in certain parts of the body. When tumours develop in the brain these often cause seizures, which are often localized in one area of the brain where the tumour is.

Epilepsy is a very common feature of TSC however many patients suffering from seizures associated with TSC are unable to obtain control of their seizures using existing AED. Alternative treatments such as surgery to remove the tumours in the brain or vagus nerve stimulation may be helpful.

Epileptic syndromes such as TSC often present with many different types of seizure. Identifying the types of seizure that a patient is suffering from is important as many of the standard AED's are targeted to treat a given seizure type these can be both generalised and focal seizure types.

The only FDA approved treatment specifically indicated for LGS, Dravet syndrome and TSC is Epidiolex® (botanically derived purified cannabidiol). Other commonly prescribed drugs include a combination of the following anticonvulsants: clobazam, clonazepam, levetiracetam, topiramate and valproic acid.

Management may also include a ketogenic diet, and physical and vagus nerve stimulation. In addition to anti-convulsive drugs, many patients with childhood onset epilepsy syndromes are treated with anti-psychotic drugs, stimulants, and drugs to treat insomnia.

Over the past forty years there have been a number of animal and human studies on the use of the non-psychoactive cannabinoid cannabidiol (CBD) to treat seizures.

A study in 1978 provided 200 mg/day of pure CBD to four adult patients, two of the four patients became seizure free, whereas in the remainder, seizure frequency was unchanged (Mechoulam and Carlini, 1978).

Cunha et al. reported that administration of CBD to eight adult patients with generalized epilepsy resulted in a marked reduction of seizures in 4 of the patients (Cunha et al., 1980) and Consroe et al., (1982) determined that CBD was able to prevent seizures in mice after administration of pro-convulsant drugs or an electric current.

In contrast to the studies described above, an open label study reported that 200 mg/day of pure CBD was ineffective in controlling seizures in twelve institutionalized adult patients (Ames and Cridland, 1986).

All of the studies described above focused on the treating subjects suffering from generalised epilepsy and did not look at the treatment of specific seizure sub-types.

The patent applications WO 2011/001169 describes the use of CBD in the treatment of focal seizures; WO 2012/093255 describes the use of CBD in combination with standard anti-epileptic drugs in the treatment of epilepsy; and WO 2013/045891 describes a composition comprising CBD and CBDV for use in the treatment of epilepsy.

Porter and Jacobson (2013) report on a parent survey conducted via a Facebook group which explored the use of cannabis which was enriched with CBD in children with treatment-resistant epilepsy. It was found that sixteen of the 19 parents surveyed reported an improvement in their child's epilepsy. The children surveyed for this paper were all taking cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported. Providing children with TRE with a cannabis extract that comprises THC, which has been described as a pro-convulsant (Consroe et al., 1977), at a potentially psychoactive dose of 0.8 mg/kg/day, is a concern.

An open label study demonstrated the use of CBD in the treatment of refractory epilepsy in the treatment of 10 patients with Tuberous Sclerosis Complex (Geffrey et al., 2014). In addition, WO 2016/059399 describes the use of CBD in the treatment of TSC at doses which were titrated up to 25 mg/kg/day.

In September 2019 the European Medicines Agency (EMA) granted marketing authorisation for the use of CBD in association with clobazam for the treatment of seizures associated with Lennox-Gastaut syndrome and Dravet syndrome in patients 2 years of age and over (European Medicines Compendium, September 2019, “Epidyolex 100 mg/ml oral solution”). Previous documents report on the adverse effects associated with CBD and clobazam in the treatment of epilepsy (Dos Santos et al., 2020; Devinsky et al. 2020; Gunning et al., 2021; Chesney et al. 2020).

The applicant has found by way of a number of double-blind placebo-controlled trials in patients with childhood onset epilepsy in three distinct syndrome, LGS, Dravet syndrome and TSC that patients that were treated concurrently with CBD and the anti-epileptic drug clobazam were at an increased risk of pneumonia. Such an interaction is unexpected and as such the use of these drugs in combination should give rise to close monitoring of the patient.

### BRIEF SUMMARY OF THE DISCLOSURE

In accordance with a first aspect of the present invention there is provided cannabidiol (CBD) for use in the treatment of childhood onset epilepsy in patients who are concurrently taking clobazam characterised in that the patient is monitored for incidence of pneumonia.

Preferably the dose of CBD is between 5 and 50 mg/kg/day. More preferably the dose of CBD is reduced by between 10% and 90%.

In a further embodiment the dose of clobazam is between 5 and 60 mg/day. Preferably the dose of clobazam is reduced by between 10% and 90%.

In one embodiment of the invention the CBD is in the form of a highly purified extract of cannabis which comprises at least 95% (w/w) CBD.

Alternatively, the CBD is present as a synthetic compound.

Preferably the childhood onset epilepsy is taken from the group consisting of: Lennox-Gastaut syndrome; Dravet syndrome; and tuberous sclerosis complex (TSC).

In a further embodiment of the invention the patient is administered antibiotic therapy in addition to the CBD and clobazam.

In accordance with a second aspect of the present invention there is provided a method of treating childhood onset epilepsy in an individual in need thereof, comprising administering to the patient a therapeutically effective amount of cannabidiol with caution, wherein the individual is concurrently taking clobazam.

Preferably the caution comprises lowering the dose of cannabidiol and/or lowering the dose of clobazam.

More preferably said caution comprises monitoring said individual for adverse events. More preferably still the adverse events are pneumonia.

In a further embodiment the caution further comprises discontinuing cannabidiol if said adverse events are observed.

Preferably the caution comprises advising said individual of side effects from said concurrent therapy.

### Definitions

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

### DETAILED DESCRIPTION

### Preparation of Highly Purified CBD Extract

The following describes the production of the highly-purified (>95% w/w) cannabidiol extract which has a known and constant composition.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD. Although the CBD is highly purified because it is produced from a cannabis plant rather than synthetically there is a small number of other cannabinoids which are co-produced and co-extracted with the CBD. Details of these cannabinoids and the quantities in which they are present in the medication are as described in Table A below.

TABLE A
Composition of highly purified CBD extract

| Cannabinoid | Concentration |
|---|---|
| CBD | >95% w/w |
| CBDA | NMT 0.15% w/w |
| CBDV | NMT 1.0% w/w |
| Δ⁹ THC | NMT 0.15% w/w |
| CBD-C4 | NMT 0.5% w/w |

>—greater than
NMT—not more than

### Preparation of Botanically Derived Purified CBD

The following describes the production of the botanically derived purified CBD which comprises greater than or equal to 98% w/w CBD and less than or equal to other cannabinoids was used in the open label, expanded-access program described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table B and the CBD API is described in Table C.

TABLE B
CBD botanical raw material specification

| Test | Method | Specification |
|---|---|---|
| Identification: | Visual | Complies |
| A | TLC | Corresponds to standard |
| | | (for CBD & CBDA) |
| B | HPLC/UV | Positive for CBDA |
| C | | |
| Assay: | In-house | NLT 90% of assayed |
| CBDA + CBD | (HPLC/UV) | cannabinoids by peak area |
| Loss on Drying | Ph.Eur. | NMT 15% |
| Aflatoxin | UKAS method | NMT 4 ppb |
| Microbial: | Ph.Eur. | NMT10⁷ cfu/g |
| TVC | | NMT10⁵ cfu/g |
| Fungi | | NMT10² cfu/g |
| E. coli | | |
| Foreign Matter: | Ph.Eur. | NMT 2% |
| Residual Herbicides | Ph.Eur. | Complies |
| and Pesticides | | |

TABLE C
Specification of an exemplary botanically derived purified CBD preparation

| Test | Test Method | Limits |
|---|---|---|
| Appearance | Visual | Off-white/pale yellow crystals |
| Identification A | HPLC-UV | Retention time of major peak corresponds |
| | | to certified CBD Reference Standard |
| Identification B | GC-FID/MS | Retention time and mass spectrum of |
| | | major peak corresponds to certified |
| | | CBD Reference Standard |
| Identification C | FT-IR | Conforms to reference spectrum for |
| | | certified CBD Reference Standard |
| Identification D | Melting Point | 65-67° C. |
| Identification E | Specific | Conforms with certified CBD Reference |
| | Optical | Standard; −110° to −140° |
| | Rotation | (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | GC | NMT 0.5% w/w |
| Alkane | | NMT 0.5% w/w |
| Ethanol | | |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a) Growing
b) Direct drying
c) Decarboxylation
d) Extraction-using liquid CO₂
e) Winterization using ethanol
f) Filtration
g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a) Crystallization using C₅-C₁₂ straight chain or branched alkane
b) Filtration
c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes adverse event profile in patients with seizures associated with LGS, DS and TSC which were recorded as part of a randomized, double-blind, parallel-group, trials of CBD versus placebo.

### Example 1: Adverse Event Profile in Patients with Seizures Associated with Lennox-Gastaut Syndrome (LGS), Dravet Syndrome (DS) and Tuberous Sclerosis Complex (TSC)

### Study Design

CBD is indicated for the treatment of seizures associated with Lennox-Gastaut syndrome (LGS), Dravet syndrome (DS) and tuberous sclerosis complex (TSC) in patients 1 year of age and older.

Randomized, double-blind, parallel-group, trials of CBD versus placebo were undertaken and the adverse event experienced by the patients were collated and investigated.

In summary patients completed a 1-week screening period and a 4-week baseline period before they were randomized to receive CBD or equivalent volumes of placebo. Randomization was stratified by age.

Patients began a 4-week dose escalation period, titrating up to their assigned dose, before continuing on a stable dose of blinded investigational medicinal product (IMP) for 12 or 14 weeks.

Dose escalation for each patient was subject to the investigator's assessment of safety and tolerability. If a dose became poorly tolerated, the investigator may consider temporarily or permanently reducing the dose for the remainder of the study.

Clinic visits occurred for screening (Day-35), baseline (Day-28), randomization (Day 1) and, on Days 15 and 29 during titration and on Days 43, 57, 71 (telephone) and 85 until the end of treatment (Day 113). Safety telephone calls were completed every two days during titration, one week after the end of titration and, for patients not entering the open label extension (OLE), weekly from Visit 10 to Visit 12.

Patients were required to perform daily interactive voice response system (IVRS) telephone calls to record seizure information. They were also required to complete a paper diary daily with information about IMP and concomitant AED administration.

Following completion of the blinded phase, patients were invited to continue to receive CBD in an OLE.

Those patients opting not to enter the OLE completed a 10-day taper period (down-titrating 10% per day for 10 days).

The OLE consisted of a 3-week titration period followed by a maintenance period and a 10-day taper period.

Following titration according to the titration schedule, patients will continue with their optimal CBD dose. However, investigators may decrease the dose if a patient experiences intolerance or increase the dose to a maximum of 50 mg/kg/day if required for better seizure control, until the optimal dose was found.

Safety and tolerability measures that were recorded included: adverse events; clinical laboratory parameters; 12-lead electrocardiogram (ECG); physical examination parameters (including height and weight); vital signs; Columbia-Suicide Severity Rating Scale (C-SSRS; 19+ years) or C-SSRS Children's (6-18 years) score, where applicable; number of inpatient hospitalizations due to epilepsy; abuse liability and effects on menstruation cycles (in females).

Patients with Lennox-Gastaut Syndrome or Dravet Syndrome

The most frequent cause of discontinuations was transaminase elevation. Discontinuation incidence for transaminase elevation was dose responsive. Risk factors for transaminase elevation include concomitant valproate and clobazam, dose of CBD, and baseline transaminase elevations.

The most common adverse reactions that occurred in CBD-treated patients with LGS or DS (incidence at least 10% and greater than placebo) were: diarrhoea; fatigue; decreased appetite; somnolence and pyrexia.

Table 1.1 below lists the adverse reactions that were reported in DS and LGS patients treated with CBD during the 14-week treatment period in the Phase 3 controlled trials.

TABLE 1.1
Adverse Reactions with a Plausible Causal Relationship to
CBD in DS and LGS Patients in Randomized Controlled Trials

| | CBD Comparative Studies | | | |
|---|---|---|---|---|
| | 10 | 20 | All | |
| | mg/kg/day | mg/kg/day | active | Placebo |
| System Organ Class | (n = 139) | (n = 307) | (n = 456^{b}) | (n = 292) |
| Preferred Term | % | % | % | % |

Gastrointestinal Disorders

| | | | | |
|---|---|---|---|---|
| Diarrhea | 13 | 21 | 18 | 10 |

General Disorders and Administration Site Conditions

| | | | | |
|---|---|---|---|---|
| Fatigue | 7 | 13 | 11 | 5 |
| Pyrexia | 17 | 15 | 16 | 12 |

Infections and Infestations

| | | | | |
|---|---|---|---|---|
| Pneumonia^{a} | 9 | 5 | 6 | 1 |

Investigations

| | | | | |
|---|---|---|---|---|
| Aspartate | 4 | 7 | 6 | 1 |
| aminotransferase | | | | |
| increased | | | | |
| Gamma-glutamyl | 4 | 5 | 4 | 2 |
| transferase | | | | |
| increased | | | | |
| Alanine | 4 | 7 | 6 | 1 |
| aminotransferase | | | | |
| increased | | | | |
| Liver function | 0 | 3 | 2 | 0 |
| test increased | | | | |
| Weight decreased | 1 | 4 | 3 | 1 |

Metabolism and Nutrition Disorders

| | | | | |
|---|---|---|---|---|
| Decreased appetite | 17 | 24 | 21 | 8 |

Nervous System Disorders

| | | | | |
|---|---|---|---|---|
| Somnolence^{a} | 26 | 29 | 29 | 10 |
| Lethargy | 3 | 6 | 5 | 2 |
| Drooling | 0 | 3 | 2 | 1 |

Psychiatric Disorders

| | | | | |
|---|---|---|---|---|
| Irritability | 7 | 5 | 6 | 2 |
| Aggression | 2 | 5 | 4 | 1 |
| Insomnia | 4 | 3 | 3 | 2 |
| Abnormal behavior | 0 | 3 | 2 | 1 |
| Agitation | 0 | 1 | 1 | 0 |

Skin and Subcutaneous Tissue Disorders

| | | | | |
|---|---|---|---|---|
| Rash | 2 | 5 | 4 | 1 |

CBD = cannabidiol; DS = Dravet syndrome; FDA = Food and Drug Administration; LGS = Lennox-Gastaut syndrome.
^{a}Grouped Terms: Pneumonia: Pneumonia, Pneumonia RSV, Pneumonia mycoplasmal, Pneumonia adenoviral, Aspiration pneumonia; Somnolence: somnolence, sedation
^{b}Includes 10 patients randomized to 5 mg/kg/day

Patients with Tuberous Sclerosis Complex

In the controlled trial in TSC, the most frequent causes of discontinuation were transaminase elevation, rash, and somnolence. Discontinuation for transaminase elevation was dose responsive.

Table 1.12 below lists the adverse reactions that were reported in TSC patients treated with CBD during the 12-week treatment period in the Phase 3 controlled trial.

TABLE 1.2
Adverse Reactions with a Plausible Causal Relationship
to CBD in TSC Patients in Randomized Controlled Trials

| | CBD | | |
|---|---|---|---|
| | 25 mg/kg/day | 50 mg/kg/day^{a} | Placebo |
| System Organ Class | N = 75 | N = 73 | N = 76 |
| Preferred Term | % | % | % |

Blood and Lymphatic System Disorders

| | | | |
|---|---|---|---|
| Anaemia | 7 | 4 | 1 |

Gastrointestinal Disorders

| | | | |
|---|---|---|---|
| Diarrhoea | 31 | 56 | 25 |
| Vomiting | 17 | 18 | 9 |
| Nausea | 9 | 3 | 3 |

General Disorders and Administration Site Conditions

| | | | |
|---|---|---|---|
| Pyrexia | 19 | 16 | 8 |
| Fatigue | 5 | 6 | 1 |

Infections and Infestations

| | | | |
|---|---|---|---|
| Urinary tract infection | 5 | 1 | 0 |
| Pneumonia^{b} | 4 | 3 | 1 |

Investigations

| | | | |
|---|---|---|---|
| Alanine aminotransferase | 12 | 22 | 0 |
| increased | | | |
| Aspartate aminotransferase | 11 | 19 | 0 |
| increased | | | |
| Gamma glutamyl transferase | 16 | 14 | 0 |
| increased | | | |
| Weight decreased | 7 | 8 | 0 |
| Eosinophil count increased | 5 | 1 | 0 |

Metabolism and Nutrition Disorders

| | | | |
|---|---|---|---|
| Decreased appetite | 20 | 23 | 12 |

Nervous System Disorders

| | | | |
|---|---|---|---|
| Somnolence | 13 | 26 | 9 |

Skin and Subcutaneous Tissue Disorders

| | | | |
|---|---|---|---|
| Rash | 7 | 12 | 4 |

^{a}The maximum recommended dosage is 25 mg/kg/day. One-third of patients randomized to the 50-mg/kg/day dosage group (2-times higher than the maximum recommended dosage) did not reach 50 mg/kg/day; adverse drug reactions listed occurred at a range of dosages up to and including 50 mg/kg/day.
^{b}Grouped Terms: Pneumonia: Pneumonia, Pneumonia RSV, Pneumonia mycoplasmal, Pneumonia adenoviral, Aspiration pneumonia

Additional Adverse Reactions in Patients with LGS, DS, or TSC

### Decreased Weight

CBD can cause weight loss. The decrease in weight appeared to be dose related. In some cases, the decreased weight was reported as an adverse event

### Hematologic Abnormalities

CBD can cause decreases in haemoglobin and haematocrit. Twenty-seven percent (27%) of CBD-OS-treated patients with LGS and DS and 38% of CBD-OS-treated (25 mg/kg/day) patients with TSC developed a new laboratory-defined anaemia during the course of the study (defined as a normal haemoglobin concentration at baseline, with a reported value less than the lower limit of normal at a subsequent time point), versus 14% of patients with LGS and DS on placebo and 15% of patients with TSC on placebo.

### Increases in Creatinine

CBD can cause elevations in serum creatinine. The mechanism has not yet been determined. In controlled studies in healthy adults and in patients with LGS, DS, and TSC, an increase in serum creatinine of approximately 10% was observed within 2 weeks of starting CBD-OS. The increase was reversible in healthy adults. Reversibility was not assessed in studies in LGS, DS, or TSC.

### Pneumonia

Pneumonia has been observed in controlled trials with clobazam (14% in patients receiving 10 mg/kg/day CBD, 7% in patients receiving 20 mg/kg/day CBD, and 1% receiving placebo) and without concomitant clobazam (0% in patients receiving 10 mg/kg/day CBD, 3% in patients receiving 20 mg/kg/day CBD, and 2% receiving placebo).

In patients with TSC receiving 25 mg/kg/day CBD, pneumonia has been observed with concomitant clobazam (17% in patients receiving 25 mg/kg/day and 0% receiving placebo) and without clobazam (0% in patients receiving 25 mg/kg/day and 2% receiving placebo).

Table 1.3 below details the incidence of pneumonia in LGS and DS patients.

TABLE 1.3
Incidence of pneumonia in LGS and DS patients

| | Cannabidiol | | | |
|---|---|---|---|---|
| | 5 | 10 | 20 | |
| | mg/kg/day | mg/kg/day | mg/kg/day | Placebo |
| Number of | N1 = 6, | N1 = 85, | N1 = 167, | N1 = 164, |
| subjects with | N2 = 4 | N2 = 54 | N2 = 140 | N2 = 128 |
| pneumonia | N (%) | N (%) | N (%) | N (%) |
| With CLB | 0 | 12 (14.1%) | 12 (7.2%) | 2 (1.2%) |
| Without CLB | 0 | 0 | 4 (2.9%) | 2 (1.6%) |

N1—Number of subjects taking clobazam,
N2—Number of subjects not taking clobazam

Table 1.4 below details the incidence of pneumonia in TSC patients.

TABLE 1.3
Incidence of pneumonia in TSC patients

| | Cannabidiol | | |
|---|---|---|---|
| | 25 | 50 | |
| | mg/kg/day | mg/kg/day | Placebo |
| Number of | N1 = 18, | N1 = 20, | N1 = 25, |
| subjects with | N2 = 57 | N2 = 53 | N2 = 51 |
| pneumonia | N (%) | N (%) | N (%) |
| With CLB | 3 (16.7%) | 1 (5.0%) | 0 |
| Without CLB | 0 | 1 (1.9%) | 1 (2.0%) |

N1—Number of subjects taking clobazam,
N2—Number of subjects not taking clobazam

As can be seen the incidence of pneumonia is significantly higher in patients that are concurrently taking clobazam in addition to CBD.

### CONCLUSIONS

These data indicate that patients that have been diagnosed with childhood onset epilepsy in three distinct syndromes: LGS, Dravet syndrome and TSC who were treated with CBD in combination with the anti-epileptic drug clobazam were at an increased risk of pneumonia. Such an interaction is unexpected and as such the use of these drugs in combination should give rise to close monitoring of the patient.

Pneumonia is a known complication of generalised tonic-clonic seizures. It is thought that pneumonia in patients with epilepsy is caused by aspiration of secretions in the nose and mouth due to the protective reflexes which normally prevent this occurring being inhibited by seizures.

Surprisingly the data presented here demonstrates that patients who had a reduction in the number of seizures experienced were at an increased risk of pneumonia. In this regard, patients who were taking CBD, either alone or in combination with clobazam experienced a significant reduction in seizure frequency versus placebo across all treatment groups as has been previously demonstrated.

It is therefore imperative that patients concurrently taking CBD and clobazam are both cautioned and monitored for occurrence of pneumonia. In patients which are at increased risk due to co-morbid lung conditions such as asthma or COPD such patients may be prescribed antibiotics to use prophylactically to prevent occurrence of pneumonia.

Furthermore, reduction of the dose of CBD, the dose of clobazam or the doses of both CBD and clobazam may be required to reduce the risk of pneumonia in patients.

1. A method of treating childhood onset epilepsy in a patient comprising administering cannabidiol (CBD) to the patient who is currently taking clobazam and monitoring the patient for incidence of pneumonia.

2. The method of claim 1, comprising administering a dose of CBD that is between 5 and 50 mg/kg/day.

3. The method of claim 2, comprising reducing the dose of CBD by between 10% and 90%.

4. The method of claim 1, wherein the dose of clobazam is between 5 and 60 mg/day.

5. The method of claim 4, wherein the dose of clobazam is reduced by between 10% and 90%.

6. The method of claim 1, wherein the CBD is in the form of a highly purified extract of cannabis which comprises at least 95% (w/w) CBD.

7. The method of claim 1, wherein the CBD is present as a synthetic compound.

8. The method of claim 1, wherein the childhood onset epilepsy is selected from the group consisting of: Lennox-Gastaut syndrome; Dravet syndrome; and tuberous sclerosis complex (TSC).

9. The method of claim 1, wherein the patient is administered antibiotic therapy in addition to the CBD and clobazam.

10. A method of treating childhood onset epilepsy in a patient in need thereof, comprising administering to the patient a therapeutically effective amount of cannabidiol with caution, wherein the patient is concurrently taking clobazam.

11. The method of claim 10, wherein said caution comprises lowering the dose of cannabidiol.

12. The method of claim 10, wherein said caution comprises lowering the dose of clobazam.

13. The method of claim 10, wherein said caution comprises lowering the dose of cannabidiol and clobazam.

14. The method of claim 10, wherein said caution comprises monitoring said patient for adverse events.

15. The method of claim 13, wherein said adverse events are pneumonia.

16. The method of claim 13, wherein said caution further comprises discontinuing cannabidiol if said adverse events are observed.

17. The method of claim 10, wherein said caution comprises advising said patient of side effects from said concurrent therapy.
